(19) United States
(12) Patent Application Publication
(10) Pub. No.: US 2003/0181989 A1
(43) Pub. Date: Sep. 25, 2003

(54) Cushioned liner and use with prosthetic devices

(75) Inventors: John D. Eberle, Amherst, NY (US); Stephen P. Schaffer, Hamburg, NY (US); Ryan Carlson, Buffalo, NY (US); Barry A. May, Lockport, NY (US)

Correspondence Address: DARBY & DARBY P.C., 805 Third Avenue, New York, NY, 10022, US

(73) Assignee: SSL AMERICAS, INC.

(21) Appl. No.: 10/102,299

(22) Filed: Mar. 19, 2002

(51) Int. Cl. A61F 2/78

(52) U.S. Cl. 623/36; 602/63

(57) ABSTRACT

An improved cushioned liner (i.e., prosthetic liner) for covering a residual limb of an amputee is provided. According to one embodiment, the cushioned liner includes a sock-shaped fabric body member formed of at least two fabric pieces. One of the fabric pieces is a distal end piece that is attached to at least one other fabric piece along a circumferential edge of the distal end piece. The distal end piece is free of a transverse seam that extends across the distal end piece. The cushioned liner also includes a cushion layer disposed on at least one side of the sock-shaped fabric member. In another embodiment, a liner body having the above construction is provided and is adapted to receive cushioning material to form the cushioned liner.

### Technical Field

The present invention relates generally to an article having a cushion layer formed on the inside and/or outside of the article (e.g., a fabric) and more particularly, to a gel cushioned liner to be worn over an amputee's residual limb.

### BACKGROUND

For the past decades, amputees have worn tubular sock-like articles over their residual limbs to provide additional comfort to the amputee when wearing a prosthetic limb. For many years, the tubular sock-like articles were formed of natural materials, such as cotton, wool, and cotton-wool blends; however, as synthetic materials become increasingly popular as a material of choice to form articles of apparel, including socks, the tubular sock-like articles were increasingly fabricated using synthetic materials.

As is known, an amputee is typically fitted with a prosthetic member to be worn over the residual limb. In a below-knee (BK) prosthesis, an amputee's stump tends to pivot within a socket of the prosthesis. During ambulation, the stump will come up in the socket of the prosthesis until the means for attaching the prosthetic to the wearer causes the prosthetic to lift with the stump. The wearer then completes a walking motion or other movement by repeatedly lifting the prosthetic up and then placing it back down in a different location to effectuate movement of the wearer's body.

Most of the available cushioned residuum socks (prosthetic liners) that are currently available have a tubular or conical construction and do not provide a form fit of the amputee's residuum since the residuum stump typically does not contain a completely uniform shape. For example, while the residuum stump generally has a roughly conical shape, the residuum stump will often have recessed areas in certain locations. On a below knee, left side residual limb, the recessed area is often more pronounced on the right side of the tibia bone, while for right side residual limbs, the more pronounced recessed area is on the left side of the bone. In both instances, the side opposite the side with the more pronounced recessed area will also contain a recessed area to a lesser degree and further the greatest recess typically occurs immediately below the patella, one either side. Conventional prosthetic liners do not accommodate the non-uniform nature of the residuum and this can result in the amputee experiencing wearing discomfort due to the non-uniform fit.

When the amputee uses a prosthetic device, the amputee simply attaches a prosthetic limb to their residual limb by means of a rigid socket, liner, and a suspension means. The rigid socket can be custom fabricated to match the shape of the intended user's residual limb and can be formed from a variety of different materials, including but not limited to thermoplastic materials, fiber-reinforced thermoset materials, as well as wood and metals. Because the residual limb interfaces with the hard, rigid prosthetic limb, this interface can become an area of discomfort over time since this interface is a load bearing interface between the residual limb and the prosthetic limb. In order to alleviate this discomfort and provide a degree of cushioning to lessen the impact of the load, prosthetic liners (socks) are used as interface members between the hard prosthetic socket and the residual limb in order to increase comfort.

One of the disadvantages of conventional prosthetic liners (socks) is that they are constructed in such a manner that the closed distal end thereof actually causes discomfort to the wearer despite being designed to provide comfort to the wearer at the interface area between the residual limb and the prosthetic limb. More specifically and as shown in FIG. 11, a conventional prosthetic liner 10 is typically constructed of two or more textile pieces 12, 14 that are stitched or otherwise attached to one another to form the sleeve-like prosthetic liner 10. At a distal end 16 of the prosthetic liner 10, the textile pieces 12, 14 are joined together along a distal seam 18. For example, the two textile pieces 12, 14 can be joined together by stitching the two pieces 12, 14 to each other along the distal seam 18. A cushioning material, generally indicated at 20, is disposed on the inside of the prosthetic liner 10, the outside of the prosthetic liner 10, or a combination thereof. The distal end 16 of the prosthetic liner 10 is the portion of the liner 10 that fits around the lower, distal section of the residual limb and acts as an interface between the residual limb and the prosthetic limb.

While the distal ends of conventional prosthetic liners, such as liner 10, are constructed to provide a comfortable interface between the residual limb and the prosthetic limb, the distal seam 18 can cause irritation to the wearer due its location across the distal end of the residual limb in a load bearing area. This discomfort results even if the textile pieces 12, 14 are interconnected using a flat-locked stitch, which is a stitch designed to provide a low, smooth seam between the two interconnected pieces, because the distal end 16 of the liner 10 bears the greatest load since it interfaces between the residual limb and the prosthetic limb and each time the wearer steps, the wearer bears down on the socket of the prosthetic limb and a load is exerted on the prosthetic limb. Because the distal seam 18 extends across the distal end of the residual limb, the distal seam 18 is constantly pressed against the residual limb each time the wearer steps. This pressing action causes irritation and discomfort to the wearer since the distal seam 18 is located in a very high load bearing area and thus, even the smoothest seam will cause discomfort because of the location of the seam 18.

Thus, there is still a need in the art for a prosthetic liner which overcomes the deficiencies of the prior art and is constructed so that the uncomfortable distal seam is eliminated, thereby increasing the comfort of the wearer.

### SUMMARY

An improved cushioned liner (i.e., prosthetic liner) for covering a residual limb of an amputee is provided. According to one embodiment, the cushioned liner includes a sock-shaped fabric body member formed of at least two fabric pieces. One of the fabric pieces is a distal end piece that is attached to at least one other fabric piece along a circumferential edge of the distal end piece. The distal end piece is free of a transverse seam that extends across the distal end piece. The cushioned liner also includes a cushion layer disposed on at least one side of the sock-shaped fabric member.

A cushioned liner having the above construction provides a number of advantages over conventional prosthetic liner constructions. More specifically, the distal transverse seam that is associated with conventional prosthetic liners has been eliminated in the present construction since the present distal seam is a circumferential seam that extends around the peripheral edge of the distal end piece instead of being formed across a medial section as in traditional prosthetic liners. By eliminating a distal seam that extends transversely across the distal end of the prosthetic liner, a wearer of the present cushioned liner experiences increased comfort due to eliminating the transverse orientation of the distal seam, which causes irritation and general discomfort, even in cases where the transverse distal seam is covered with a cushioning material.

In yet another embodiment, a liner body is provided having the above construction and is adapted to receive a cushioning material on at least one side thereof to form the above-described cushioned liner.

Other features and advantages of the present invention will be apparent from the following detailed description when read in conjunction with the accompanying drawings.

### BRIEF DESCRIPTION OF THE DRAWINGS

The foregoing and other features of the present invention will be more readily apparent from the following detailed description and drawings of illustrative embodiments of the invention in which:

FIG. 1 is a perspective view of a cushioned liner according to one exemplary embodiment being placed over a residual limb of an amputee;

FIG. 2 is a perspective view of the cushioned liner of FIG. 1 with the residual limb being fully inserted into the cushioned liner;

FIG. 3 is a partially exploded perspective view of the cushioned liner of FIG. 1 with a section of the cushioned liner being shown in cross-section;

FIG. 4 is an exploded perspective view of a liner body of the cushioned liner illustrating exemplary points of attachment between the individual components;

FIG. 5 is a perspective an exemplary fabric taken from circle 5 of FIG. 4 used to form the individual components of the liner body of FIG. 4;

FIG. 6 is a cross-sectional view taken from circle 6 of FIG. 3;

FIG. 7 is a perspective view of an exemplary cushioned liner according to another embodiment;

FIG. 8 is a perspective view of an exemplary cushioned liner according to yet another embodiment;

FIG. 9 is a partially cut away perspective view of the cushioned liner of FIG. 1 with the residual limb being partially inserted into the cushioned liner;

FIG. 10 is an enlarged view taken from circle 10 of FIG. 9 illustrating a distal end of the cushioned liner in partial cross-section and in a partially cut away manner to show the attachment of the individual components forming the liner body;

FIG. 11 is a partially cut away, partial cross-sectional perspective view of a distal end of a conventional prosthetic liner;

FIG. 12 is a side elevational view, in partial cross-section, of an apparatus for applying a cushioning material to a liner body to form the cushioned liner of FIG. 1;

FIG. 13 is an enlarged cross-sectional view illustrating a housing member having a cavity for receiving the liner body which in turn receives liquid or molten cushioning material;

FIG. 14 is a side elevational view, in partial cross-section, of the apparatus of FIG. 12 illustrating a mandrel being extended to an intermediate position inside of the liner body;

FIG. 15 is a side elevation view, in partial cross-section, of the apparatus of FIG. 14 illustrating the mandrel being further extended to a distal position; FIG. 16 is an enlarged cross-sectional view of the mandrel in the distal position of FIG. 15 with cushioning material flowing around the mandrel between the mandrel and the inside surface of the liner body;

FIG. 17 is a side elevational view of the apparatus of FIG. 12 illustrating the mandrel being retracted from the cavity with the cushioned liner of FIG. 1 surrounding the mandrel;

FIG. 18 is a side elevation view of the mandrel in the retracted position of FIG. 17 with the cushioned liner being rolled or peeled therefrom for removal of the cushioned liner;

FIG. 19 is side elevational view of an apparatus according to an alternative embodiment for applying a cushioning material to a liner body to form the cushioned liner of FIG. 1, wherein the mandrel is formed to have a non-uniform shape; and

FIG. 20 is an enlarged cross-sectional view illustrating the mandrel of FIG. 19 being inserted into a liner to form a cushion layer having a section of increased thickness.

### DETAILED DESCRIPTION OF THE PREFERRED EMBODIMENTS

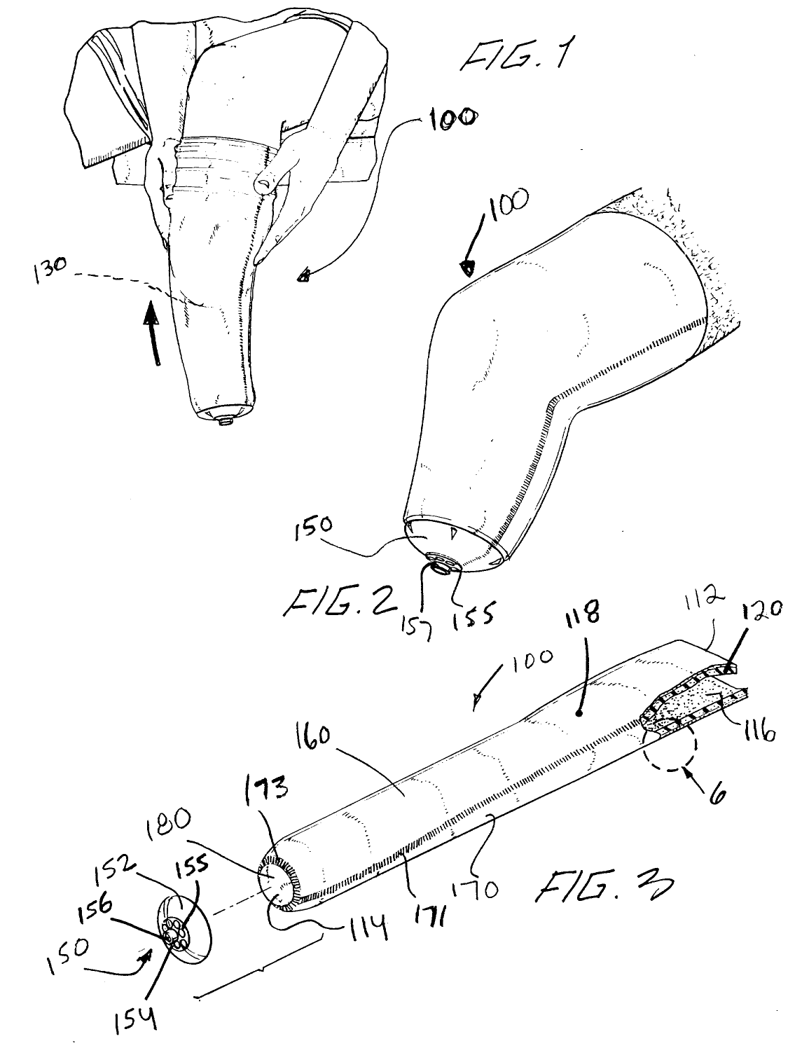

Referring to FIGS. 1 through 6, a cushioned liner (e.g., a prosthetic liner) 100 according to one exemplary embodiment is illustrated. The cushioned liner 100 is formed of a liner body 110 having a form fitting generally tubular sock-shape with an open end 112 into which an amputation stump (residual limb) 130 can be introduced and a closed distal end 114. The liner body 110 includes an interior 116 and an exterior 118 with the interior 116 being impregnated with a cushioning material to form a cushion layer 120 so as to provide a cushion between the amputee's residual limb 130 and a prosthetic device (not shown) which is to be attached to or otherwise coupled to the residual limb 130, as will be described in greater detail below.

When the cushioned liner 100 is used to couple a prosthetic device to the residual limb 130, a pin receptacle 150 is preferably provided and is attached to the distal end 114 of the cushioned liner 100 on the exterior 118 thereof. In the exemplary embodiment, the pin receptacle 150 has a resilient radial skirt portion 152 surrounding a receptacle body 154. The receptacle body 154 is a rigid member that is preferably formed of metal and includes a threaded bore 156 which threadingly receives a connecting member (e.g., a threaded pin) of the prosthetic device to securely attach the prosthetic device to the cushioned liner 100. The radial skirt portion 152 preferably has a diameter that is approximately equal to or less than the diameter of the distal closed end 114 of the liner body 110 so that the radial skirt portion 152 does not extend beyond the peripheral edge of the liner body 110 at the distal end 114 thereof. In other words, there preferably is a smooth radial interface between the radial skirt portion 152 and the liner body 110. However, in some applications, it may be desirable for the radial skirt portion 152 to extend beyond the peripheral edge of the liner body 110.

The radial skirt portion 152 is a flexible member that is formed of a resilient material, such as a polymeric material. The receptacle body 154 can be formed of any number of materials, such as metals, and in one embodiment, the receptacle body 154 is formed of aluminum.

The pin receptacle 150 is disposed at the closed distal end 114 of the liner body 110 using any number of techniques. When the pin receptacle 150 is disposed on the distal end 114, the receptacle body 154 is generally centered about the distal end 114. The receptacle body 154 has an annular base 155 (i.e., radial flange) that surrounds an annular boss 157 that includes the threaded bore 156. Preferably, the radial skirt portion 152 is formed over the receptacle body 154 and the polymeric material forming the radial skirt portion 152 surrounds the outer surface of the annular boss 157. In other words, the only portion of the receptacle body 154 that is exposed is the threaded bore 156 to receive the connecting member of the prosthetic device and establish a connection between the cushioned liner 100 and the prosthetic device.

Suitable techniques for attaching the pin receptacle 150 to the closed distal end 114 include but are not limited to using an adhesive material to bond the pin receptacle 150 to the textile material of the closed distal end 114. It will also be appreciated that a molding process can be used to form the radial skirt portion 152 around the receptacle body 154 and at the same time bond the socket 150 to the distal end 114 of the cushioned liner 100. For example, the receptacle body 154 can be placed into a mold, along with the distal end 114 of the liner body 110 and then polymeric material can be introduced into a mold cavity, thereby forming the radial skirt portion 152 and attaching the pin receptacle 150 to the liner body 110.

As best shown in the exploded view of FIG. 4, one exemplary liner body 110 is formed of several or more pieces (panels) of textile material that are cut according to an exemplary pattern and then attached to one another along predetermined seams to provide the constructed liner body 110. In one exemplary embodiment, the liner body 110 is formed of three pieces of textile material, namely first and second side panels 160, 170 and a distal panel 180. Preferably, the first and second side panels 160, 170 are identical to one another. Each of the first and second side panels 160, 170 has an upper edge 162 that forms the open end 112 of the liner body 110 when the first and second side panels 160,170 are attached and an opposing lower edge 164 that forms the closed distal end 114 of the liner body 110.

When each of the first and second side panels 160, 170 is flattened out, each panel has a generally rectangular shape with a slight inward taper toward the lower edge 164. In other words, the upper edge 162 has a width slightly greater than the width of the lower edge 164. Each of the first and second side panels 160, 170 has an interior surface 172 (that forms a part of the interior 116 of the liner body 110) and an opposing exterior surface 174 (that forms a part of the exterior 118 of the liner body 110). As best shown in FIG. 4, the first and second side panels 160, 170 are attached to one another along side edges 166 of each. The side edges 166 extend from the lower edge 164 to the upper edge 162.

The distal panel 180 is a textile piece that is cut to have an annular shape or some other desired shape so long as the distal panel 180 encloses one end of the liner body 110 when it is connected to the side panels 160, 170. The distal panel 180 has an interior surface 182, an exterior surface 184 and a peripheral, circumferential edge 186. The dimensions of the distal panel 180 should be such that when the first and second side panels 160, 170 are attached to one another, the distal panel 180 completely extends across the open lower edge (i.e., defined by the lower edges 162 of the panels 160, 170) so as to enclose the distal end (second end 114) of the liner body 110. Accordingly when the first and second side panels 160, 170 are attached to one another along the side edges 166 to form vertical seams, the liner body 110 has a tubular shape and the distal panel 180 is used to enclose the liner body 110. The distal panel 180 is attached to the lower edges 162 of the first and second side panels 160, 170 along its peripheral, circumferential edge 186.

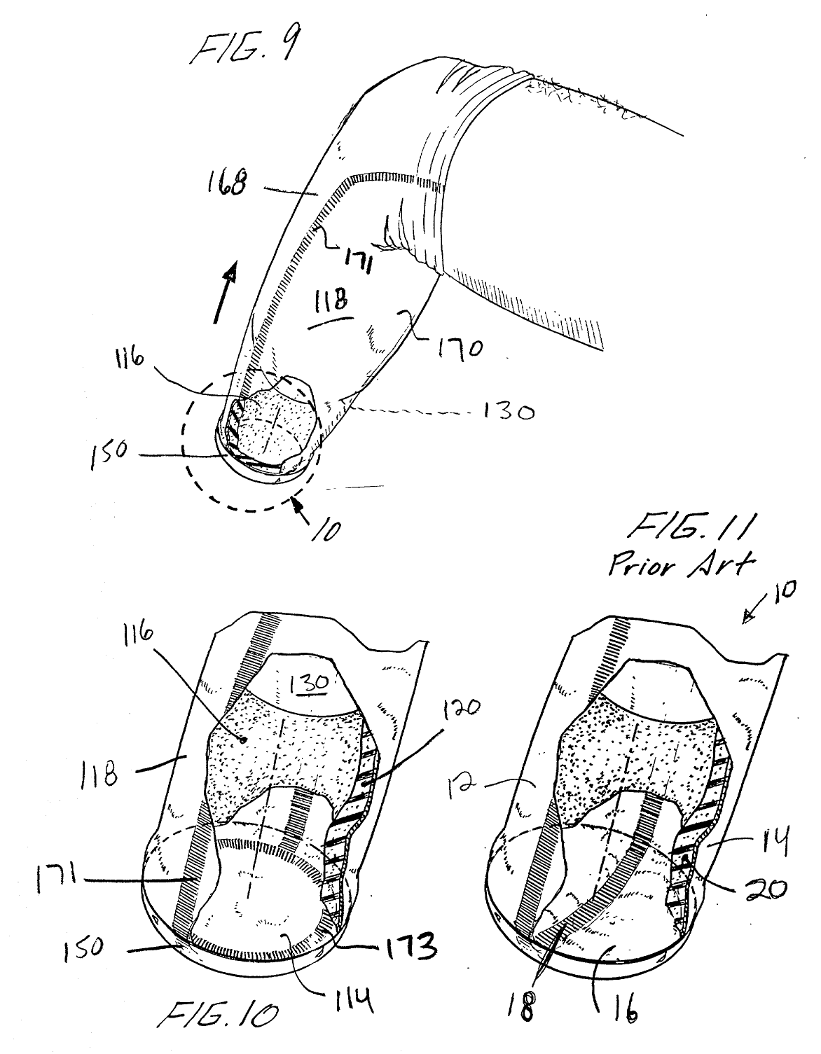

As best shown in FIG. 5 and according to one embodiment, the interior surfaces 172, 182 of the first and second side panels 160, 170 and the distal piece 180, respectively, have a different texture than the exterior surfaces 174, 184. As will be described in greater detail hereinafter, the textile panels 160, 170, 180 are preferably formed of two different materials that are knit together so that the fibers of one material form the exterior surface of the respective piece and the fibers of the other material form the interior surface of the respective piece. The texture of the interior surfaces 172, 182 is designed to absorb the cushioning material that is applied to the interior surfaces 172, 182 to form the cushion layer 120, while not permitting the cushioning material to bleed through or otherwise migrate to the exterior surfaces 174, 184 thereof. As illustrated in FIG. 5, the exemplary interior surface of the textile material has a waffle-like appearance for absorbing the cushioning material. It will be understood that the liner body 110 can be formed of other fabric materials having different textures that the aforementioned textures. For example, the texture of each side of the liner body 110 can be the same.

Referring now to FIGS. 1 through 8, the two side panels of material 160, 170 used to construct the liner body 110 can be attached to one another using any number of conventional techniques, including stitching the two side panels 160, 170 of textile material along the side edges 166 to form vertical stitched seams 171. When the two side panels 160, 170 are stitched to each other, a wide variety of thread types can be used and a number of different stitch types can be used. In one exemplary embodiment, thread formed of a synthetic material, such as nylon, is used to attach the two side panels 160, 170 to one another using a flat-locked stitch. A flat-locked stitch is preferred because this type of stitch tends to create a smooth seam that is less irritating than seams formed of other stitches. A flat-locked stitch also permits the two side panels 160, 170 to sufficiently stretch to accommodate the stretching of the cushioned liner 100 that occurs during the normal wear of the cushioned liner 100.

Similarly, the distal panel 180 of textile material can be connected to the distal lower ends 164 of the two side panels 160, 170 along a circumferential stitched seam 173 using any number of stitch types. However, the distal panel 180 of material is preferably connected to the distal lower ends 164 of the first and second side panels 160, 170 of material using a circumferential seam 173 that has a flat-locked stitch.

The first and second side panels of material 160, 170 and the distal panel 180 can be formed of any number of different textile materials having a predetermined thickness (ply). Preferred textile materials are textile fabrics that have an elasticity that permits the prosthetic liner (cushioned liner 100) to stretch a predetermined amount during normal application of the cushioned liner 100 to the residual limb 130 and during the normal motions of the cushioned liner 100 as the wearer takes steps or otherwise moves the prosthetic limb (i.e., the prosthetic device). For example, the two side panels 160, 170 and the distal panel 180 can be formed of fabrics selected from the group consisting of: stretchable non-woven fabrics (e.g., the Xymide line of fabrics including Wearforce® fabrics from DuPont, Wilmington, Del.); Lycra® based materials which include segmented elastomeric polyurethane fibers (i.e., spandex type fabrics); supplex nylon, neoprene fabrics (polychloroprene fabrics); nylon, spunbonded olefin; looped nylon; spunlaced fabrics; polyester; polypropylene; and aramid fiber fabrics. It will be appreciated that the above list of suitable fabric materials is not exhaustive and is merely exemplary in nature and not limiting of the types of fabric materials that be used to form the liner body 110. Further, it will be appreciated that the fabrics used to form the present liner body 110 are preferably elastic fabrics that can be provided in a woven, knitted, or non-woven form.

One preferred fabric material that is used to form the two side panels 160, 170 and the distal panel 180 is a fabric formed of polyester and polypropylene knit fibers. As shown in FIG. 5, the fabric is constructed (knit) in such a way that the polyester fibers form one surface of the fabric and the polypropylene fibers form the opposite surface of the fabric. In the present prosthetic liner, the polyester surface is intended to form a part of the exterior 118 of the liner body 110, while the polypropylene surface is intended to form a part of the interior 116 of the liner body 110. The polypropylene surface has a distinct texture in that it has a waffle-like texture. When the fibers are knitted in this manner (waffle-like), a number of interstices are formed across the polypropylene surface. As will be described in greater detail hereinafter, the interstitial characteristic of the side of the fabric that forms the interior of liner body 110 advantageously permits gel that is applied to the interior of the liner body 110 to be readily absorbed within the interstices. This type of fabric is commercially available from Milliken & Company of Spartanburg, S.C. under the style/pattern No. 952561-804. Advantageously, a fabric constructed in the aforementioned manner allows the cushioning material to enter into the denure of the fabric but is resistive to the cushioning material migrating through the fabric from the interior surface 116 across the exterior surface 118. In the final product, the cushioning material should be confined to the interior surfaces 116, while the exterior surfaces 118 are free from the cushioning material.

The material used to form the liner body 110 is preferably elastic (stretchable) in one or more, preferably two, directions and is capable of adjusting to variations in form and size of the residual limb 130. Depending upon the precise application, the thickness of the textile material (e.g., fabric) can be altered and while in one embodiment, the material/material thickness of each of the first and second side panels 160, 170 is the same as the material/material thickness of the distal panel 180, it will be appreciated that the material and/or material thickness of any one of the first and second side panels 160, 170 and the distal panel 180 can be different from the other pieces. In one exemplary embodiment, the thickness of the fabric material used to construct the liner body 110 is from about 0.010 inch to about 0.200 inch. In the embodiment where the fabric material is a knit of polyester and polypropylene fibers, the thickness of the fabric material is about 0.04 inch; however, the thickness of the polyester/polypropylene knit can vary depending upon the particular application.

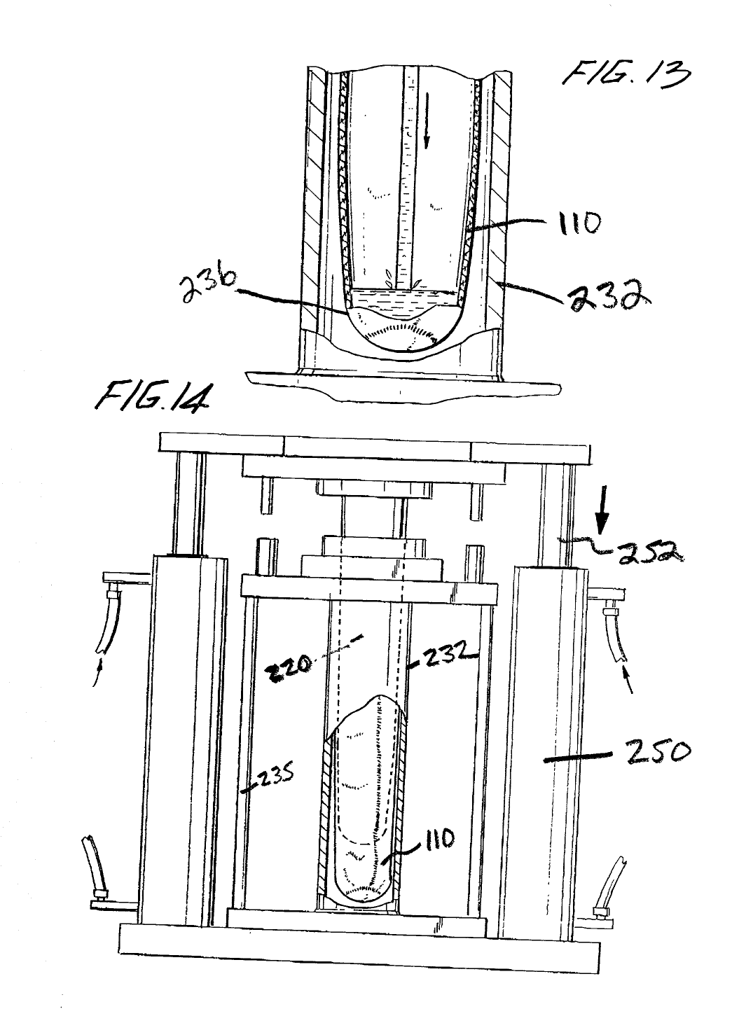

As best shown in FIGS. 9 through 11, the cushioned liner 100 of FIGS. 9 and 10 provides a number of advantages over the conventional prosthetic liner 10 shown in FIG. 11. More specifically, the distal seam 18 of the traditional prosthetic liner 10 has been eliminated by constructing the present cushioned liner 100 so that it includes a distal panel 180 at the distal end 114 that is attached to the two side fabric panels 160, 170 along the circumferential seam 173 that extends around the peripheral edge of the distal panel 180 instead of being formed across a medial section as in the traditional prosthetic liner 10. By eliminating a distal seam that extends across the distal end of the prosthetic liner, such as distal seam 18, the wearer of the present cushioned liner 100 experiences increased comfort since the distal seam 18 is associated with irritation and general discomfort, even in the cases where the distal seam 18 is covered with a cushioning material.

It will be appreciated that the distal end 114 of the cushioned liner 100 does not include a seam that is positioned in a location where the residual limb 130 will come into contact therewith. In many cases, the residual limb 130 tapers inwardly toward its distal stump end due to the natural shape of a leg and as a result of typical surgical techniques that are employed during an amputation procedure. The residual limb 130 thus rests against the cushion layer 120 in an area that is within the circumferential seam 173 or at least preferably contacts the circumferential seam 173 at the most peripheral portions of the residual limb 130. At the very least, the wearer of the present cushioned liner 100 does not experience a distal seam running across underneath the residual limb 130 and preferably, the cushioned liner 100 is constructed so that the contact between the residual limb 130 and the circumferential seam 173 is negligible or nonexistent. As previously-mentioned, this distal end of the residual limb 130 is an extremely sensitive area and therefore, the elimination of any stitching across this sensitive area, provides a cushioned liner that is substantially more comfortable than traditional prosthetic liners.

The cushioning material is applied to the interior surfaces 172, 182 of the first and second side panels 160, 170 and the distal panel 180, respectively, to form the cushion layer 120. The process of applying the cushioning material and controlling the thickness of the cushioning material, so as to permit contouring of the cushioning material, along the interior surface 116 of the liner body 110, will be described in greater detail below. Preferably, the cushioning material is applied to interior surfaces 172 of the first and second side fabric panels 160, 170 to coat these panels from the lower edge 164 to the upper edge 162 and is also applied to the interior surface 182 of the distal panel 180.

As previously-mentioned, the cushioned liner 100 includes the layer 120 of cushioning material and has a form fitting shape with an open end 112 into which the amputation stump 130 may be introduced, a closed end opposite the open end, an interior and an exterior. The interior of the cushioned liner 100 is defined by the interior surfaces 172, 182 of the panels 160, 170, 180 that are attached to one another and the interior surfaces 172, 182 are impregnated with a cushioning material to provide a cushion (e.g., cushion layer 120) between the amputee's residuum 130 and any prosthetic device to be worn, attached to, etc., the residuum 130.

The cushioning material is preferably a polymeric material and in one exemplary embodiment, the cushioning material is formed of a gel, a thermoplastic elastomer, or a combination thereof. For example, suitable thermoplastic elastomers include but are not limited to thermoplastic rubbers, silicon containing elastomers, thermoformable materials, etc., that provide a comfortable interface between the residuum 130 and a prosthetic device.

In one exemplary embodiment, the cushioning material is a polymeric gel that is composed of a block copolymer and mineral oil. The gel that can be used to form the cushioning material can either be a nonfoamed gel or a foamed gel (which is produced using a foaming agent). The mineral oil is present in an amount that is effective to produce a cushioning material having desired properties and is preferably present in from 0-85% by weight based on total weight, depending upon the precise application.

The polymeric material used to form the cushioned liner 100 is characterized by a certain durometer range. According to one exemplary embodiment, durometers for the cushioning material range from 1-20 on the Shore “A” scale. The lower the Shore A number, the softer the material, typically due to a higher level of plasticizer. Preferably the polymeric gel has a durometer (Shore A) that matches or approximates human skin and it has been found that the above durometer range of 1-20 generally provides the gel material with suitable characteristics. In one embodiment, the mineral oil is present on an equal weight basis, or in a weight ratio of 1/4, with regard to the amount of polymeric material present. The mineral oil is preferably purified mineral oil and is preferably USP grade.

In one exemplary embodiment, the cushioning material is formed of a Kraton®-type rubber material (Shell Chemical Co.). For example, the polymeric material can be formed of the following Kraton® rubbers: styrene-ethylene/butylene-styrene block copolymers or styrene-ethylene/propylene block copolymers and are available in triblock and diblock form.

The polymeric cushioning material can also be a blend of Kraton® rubbers and oils, such as mineral oils, (including typical stabilizers) which provide an average durometer of from 1-20. These blends typically are formed of a rubber having a lower durometer (1-10 of the Shore “A” scale) and a rubber having a higher durometer (e.g., 11-20). The blends are preferably capable of being stretched 100% or more before tearing and are capable of providing a form fit to the residual limb due to their inherent elasticity. Further, low durometer Kraton® rubbers and other materials tend to provide the cushioning material disposed of the interior 116 of the cushioned liner 100 with a sticky feeling which enhances the ability of the cushioned liner 100 to be form fitted against the residual limb 130 due to the intimate contact between the cushioning material and the skin.

In one exemplary embodiment, the polymeric material is a styrene isoprene/butadiene block copolymer or styrene-ethylene/butadiene-styrene block copolymer. Suitable polymeric materials, having the aforementioned desired properties, are commercially available from a number of sources. For example, polymeric materials commercially distributed under the trade names C-Flex 1970-W5 (R70-339-000), C-Flex 1960-W5 by Consolidated Polymer Technologies of Largo, Fla. and under the trade name Kraton G1654 by Shell Chemical Co. are suitable for use in producing the cushioning material.

The ratio of polymer to mineral oil will vary depending upon the precise application and upon the desired characteristics of the cushioned liner 100. Generally, the ratio of polymer to mineral oil can be from about 1:1 to about 4:1. In addition to using styreneisoprene/butadiene or styreneethylene/butadiene-styrene block copolymers (mixed with mineral oil), other suitable polymeric materials include styrene-butadiene-styrene and any thermoplastic elastomer or thermoformable material that is capable of being blended with mineral oil and can perform the prescribed function of providing a cushioning material suitable for use in the intended applications. Mixtures of all of the aforementioned polymers can also be used to form the polymeric cushioning material.

In one preferred exemplary embodiment, the cushioning material is a polymeric material that has gel-like characteristics and is formulated as a blend of a polystyrene-poly(ethylene-ethylene/propylene)-polystyrene block copolymer (SEEPS) and oil, such as one or more mineral oils. A suitable gel-like cushioning material formed of a SEEPS copolymer/mineral oil blend is commercially available under the trade name PolyGel 51299 from PolyGel LLC of Whippany, N.J.

The cushioning material is also selected so that the cushioned liner 100 can be placed on the residual limb 130 in such away that the polymeric material does not drag against the skin. For example, it is desirable for the cushioned liner 100 to be capable of being rolled before the cushioned liner 100 is placed on the residual limb 130 and/or prosthetic device. Advantageously, the cushioning material is also designed to provide beneficial moisture to the residual limb 130 during the wearing of the cushioned liner 100. Moreover, the cushioning material may include antioxidants, such as vitamins A, B, and C or any other antioxidants commonly used in polymers. In addition, skin conditioning agents can be added to the polymeric material of the cushioned liner 100 to soothe the skin of the residuum during wear. Such skin conditioners include mineral oil, baby oil, etc., which can be added to the polymeric material prior to its application to the liner body. Also, astringents, biocides, medicaments, etc., can be added or applied to the cushioning material to avoid infection or heal sores, etc.

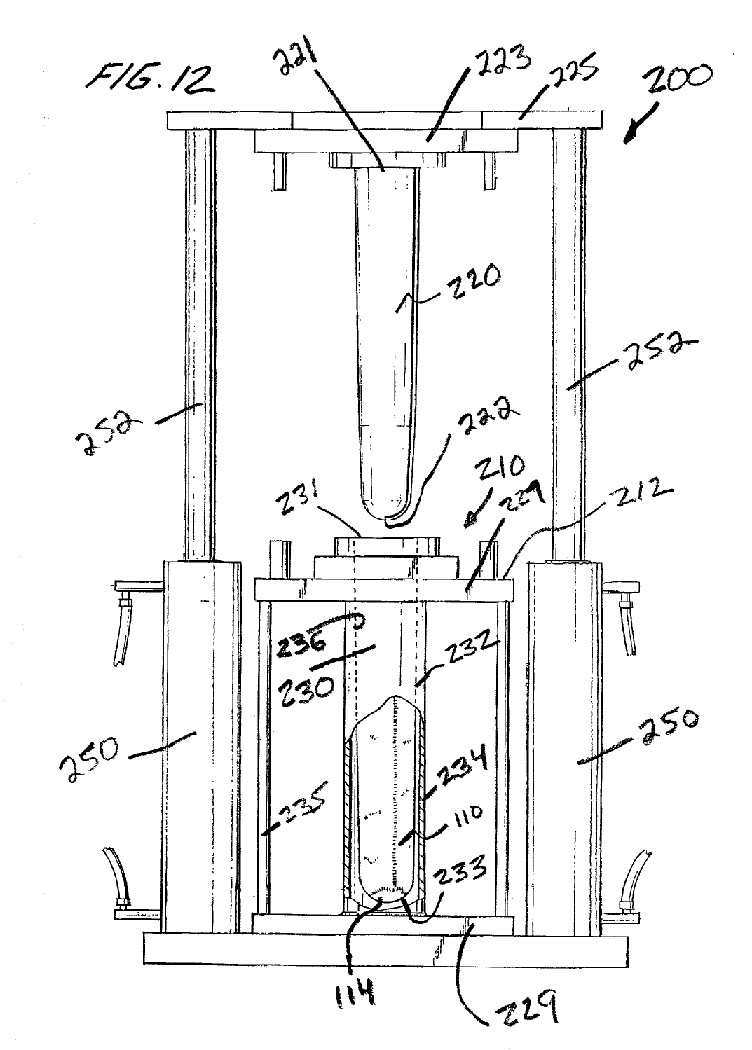

FIGS. 7 and 8 illustrate alternative embodiments for forming the cushioned liner 100. In both of the illustrated embodiments, a feature is incorporated into the cushioned liner 100 for limiting the elasticity of the cushioned liner at its distal end portion. More specifically, it is preferred to limit the elasticity of the cushioned liner 100 at the distal end portion since during normal use, the wearer is able to feel the cushioned liner stretch at the distal end portion. This stretching can lead to an uncomfortable feel as the wearer is walking or otherwise in motion. It is therefore desirable to limit the degree of elasticity at the distal end portion by adding a simple yet effective “anti-stretch” feature to the cushioned liner 100.

In the embodiment of FIG. 7, this is accomplished by adding embroidery 111 to one or more sections of the liner body 110 prior to the cushioning material being applied thereon. For example, the embroidery 111 can be in the form of vertical stitching that extends along a length of the distal end portion of one of the first and second fabric side panels 160, 170 of the liner body 110. The embroidery 111 functions as “anti-stretch” feature due to the embroidery 111 itself having a very low elasticity value. Preferably, the embroidery 111 extends completely to the lower end 162 of the one lateral piece 160, 170 and in one embodiment, the length of the embroidery 111 is between about 3 inch and about 7 inch (however, the aforementioned lengths are merely exemplary and not limiting).

While the embroidery 111 can be located anywhere along the distal end portion of one of the side fabric panels 160, 170, the embroidery 111 is preferably formed on top of one of the side seams 171. The side seams 171 represent sections of the liner body 110 where the elasticity in the longitudinal direction is already reduced or limited due to the very fact that the side seams 171 are formed of stitching (which limits the elasticity of the liner body 110 along the seam length). The embroidery 111 thus serves to supplement the elasticity limiting characteristics of the side seam 171 and reduces the overall longitudinal elasticity of the distal end portion.

In one embodiment, two distinct sections of embroidery 111 are formed along the distal end portion and are spaced about 180° apart from one another. Preferably, the two sections of embroidery 111 are formed on the two side seams 171, which are orientated about 180° from one another. However, it will be understood that more than two embroidered sections 111 can be formed at the distal end portion and that the embroidery 111 is not limited to being formed on the side seams 171. For example, four embroidered sections 111 can be formed and spaced at predetermined intervals from one another (e.g., 90° apart from one another). FIG. 7 illustrates one type of stitch pattern that is used to form the embroidered section 111; however, any number of different stitch patterns can be used to form the embroidered section 111. By varying the stitch pattern, the elasticity at the distal end portion is likewise varied. In other words, some intricate stitch patterns result reduce the elasticity of the distal end portion greater than other stitch patterns that have more simpler configurations.

FIG. 8 illustrates yet another manner of incorporating a feature for limiting the elasticity of the cushioned liner at its distal end portion. In this embodiment, the “anti-stretch” feature is in the form of a anti-stretch element (i.e., an elongated anti-stretch element). In one embodiment, one or more strips 113 of non-stretch type material (i.e., nylon) can be attached longitudinally to one of the first and second fabric side panels 160, 170 at the distal end portion of the liner body 110. This type of material acts as an “anti-stretch” feature as a result of the strip material having a low elasticity value. In one embodiment, the strip 113 is formed of a hook type material, a loop type material, or a combination thereof or the anti-stretch element can be in the form of nylon webbing.

As with the embodiment of FIG. 7, the length of each strip 113 is preferably between about 3 inch and 7 inch; however these lengths are merely exemplary and not limiting. In one embodiment, two distinct strips 113 are disposed along the distal end portion and are spaced about 180° apart from one another. Preferably, the two strips 113 are formed on the two side seams 171, which are orientated about 180° from one another. However, it will be understood that more than two strips 113 can be formed at the distal end portion. For example, four strips 113 can be formed and spaced at predetermined intervals from one another (e.g., 90° apart from one another with two being placed over the side seams 171).

The size of the cushioned liner 100 can be varied depending upon the dimensions of the residual limb to be enclosed by simply proportionally varying the dimensions of the pattern which is used to cut and form each of the first and second side panels 160, 170 and the distal panel 180. In other words, the length of the cushioned liner 100 of any of the embodiments disclosed herein can vary and the cushioned liner 100 can easily be manufactured in a number of different sizes by simply altering the dimensions of the patterns used to form the first and second fabric side panels 160, 170 and the distal piece 180. In one exemplary embodiment, the cushioned liner 100 has a length between about 8 inch to about 20 inch. Typically, the cushioned liner 100 is constructed to have a prescribed length and then the individual wearer's can modify the length of the cushioned liner 100 by simply cutting and removing an upper portion of the article. In this manner, the cushioned liner 100 can be initially produced to have a length that fits or can be easily modified to fit a large percentage of the potential customers.

As will be described in great detail hereinafter, the thickness of the cushioning material can vary along the interior surfaces 172, 182 of the first and second side fabric panels 160, 170 and the distal panel 180, thereby permitting thickness variations in prescribed areas where additional cushioning is desired to provide added comfort and protection or where less cushioning is desired due to other practical considerations. The overall thickness of the cushioned liner 100, that is the sum of the thickness of the textile liner body 110 and the thickness of the cushion layer 120, is between about 2 mm and 19 mm, according to one exemplary embodiment. It will be understood that the foregoing measurements are merely illustrative and do not limit the scope of protection for the cushioned liner 100. Thus, there may be particular applications where a thickness outside of the aforementioned range may be desired.

Several methods can be used to apply the cushioning material to the liner body. One method uses conventional dipping techniques in which the closed distal end of the liner body is dipped into the cushioning material which exists in a liquified or molten state. The liner body is dipped into the cushioning material at a prescribed angle relative to the surface of the molten or liquified cushioning material so that the cushioning material extends up the liner body from the closed distal end to a further extent on the side of the liner body. The liner body is then manipulated in the liquified or molten cushioning material to effectively coat the surface of the liner body with the cushioning material. When the cushioning material is applied in this manner, the liner body has likely been inverted so that the interior surface is actually the exterior surface that is exposed to the liquified or molten cushioning material. After application of the cushioning material by dipping the inverted liner body into the liquified or molten cushioning material and permitting the cushioning material to sufficiently cool, the coated liner is then inverted again so that the surface that has the cushioning material applied thereon becomes the interior surface of the cushioned liner.

Prior to inserting (i.e., dipping) the liner body into the liquified or molten cushioning material, a mandrel or the like is inserted into the inverted liner body to stretch and shape the liner body to its intended tubular shape. The mandrel is thus a tool that permits a person to dip the liner body into the liquified or molten cushioning material without exposing the person to any unnecessary risks. The mandrel is then manipulated so that the exposed surfaces of the liner body are in contact with the cushioning material. In order to increase the thickness, the liner body can be repeatedly dipped so as to effectively build-up the thickness of the cushioning material.

One of the disadvantages of the dipping method is that control of the thickness of the cushioning material is rather an arduous task and marked by a level of imprecision due to repeatedly dipping the liner body to build-up the coating. Further, the liner body must be inverted before and after the cushioning material is applied to the liner body. After the cushioning material has been applied and allowed to cool, the final inversion of the cushioned liner can cause folding, crazing, or other imperfections to form in the layer of the cushioning material.

In addition to the application of the cushioning material to the liner body by dipping the liner body into liquified or molten cushioning material, the cushioning material can be “painted” onto the liner body or it is also possible to dissolve the polymeric material in a solvent followed by application of the solvent to the liner body with subsequent evaporation of the solvent, thereby leaving a layer of cushioning material formed on the liner body. This method is also marked by a degree of imprecision relative to forming the cushioning material to a desired thickness.

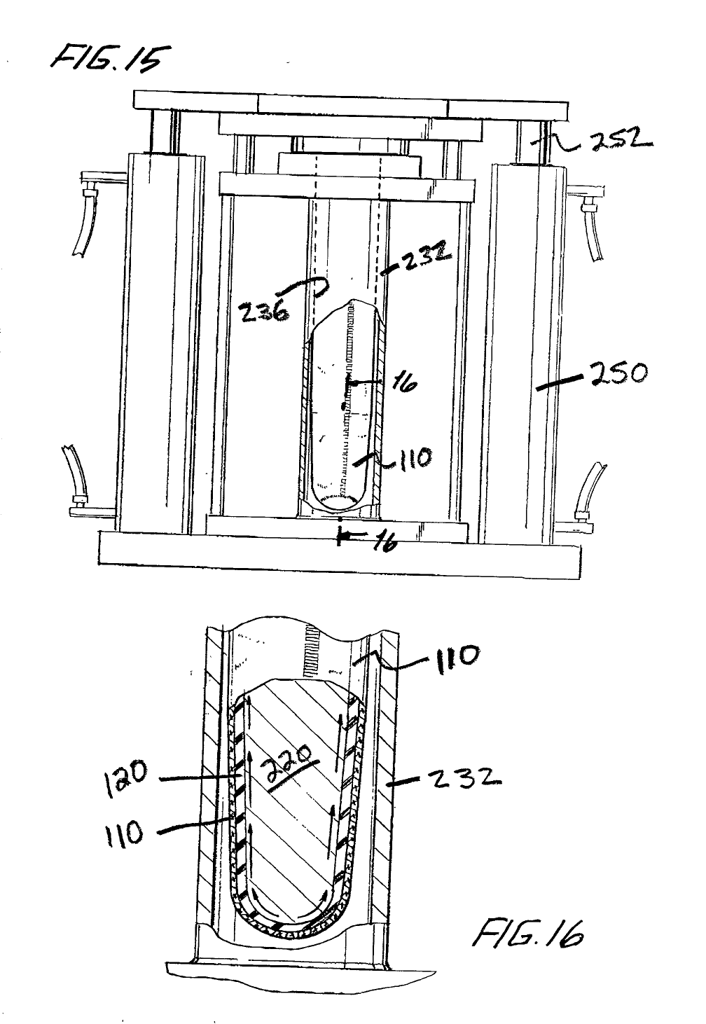

Yet another method of applying the cushioning material to the liner body 110 is illustrated in FIGS. 12 through 20. In this embodiment, the cushioning material is applied under pressure and is done in such a way that the thickness of the cushioning material can be controlled to a much greater degree of precision compared to conventional methods of applying the cushioning material, including the aforementioned dipping process. The thickness of the cushioning material can also be varied in select regions of the liner body so as to provide additional or less cushioning in the selected regions.

Another method for applying the cushioning material is commonly referred to as an “open pour” process in which the cushioning material is poured into a form and the material settles due to gravitational forces. The disadvantages of this process are that it offers little or no precise control of the thickness of the article and also a poor bond results between the fabric and the material.

An apparatus 200 for applying the cushioning material under pressure to the interior 116 of the liner body 110 is illustrated in FIG. 12. The apparatus 200 includes a base member 210 and a positionable mandrel 220. The base member 210 has a cavity 230 formed therein and has a predetermined shape and predetermined dimensions that can be varied according to the precise application. The base member 210 has an upper surface 212 that is preferably planar in nature and the cavity 230 is defined by a surrounding structure 232, which in the illustrated embodiment is a vertical housing that has a circumferential outer surface 234 and an inner surface 236 (cavity wall) that defines the cavity 230. The vertical housing 232 thus has an open end 231 and an opposing closed second 233.

In the exemplary embodiment, the base member 210 includes a first plate 237 which defines the upper surface 212 and a spaced second plate 229 with a number of legs 235 extending between the first and second plates 227, 229 to support the first plate 227 relative to the second plate 229. The vertical housing 232 extends between the first and second plates 227, 229 and can extend through complementary openings formed in one or more of the first and second plates 227, 229. The vertical housing 232 preferably is arranged in a central location of both of the first and second plates 227, 229 such that the legs 235 are located radially around the vertical housing 232. It will be understood that a number of different constructions are possible for the base member 210 so long as the base member 210 includes the cavity 230. The base member 210 can be formed of a number of materials so long as the material that defines the cavity 230 can be exposed to the elevated temperatures that are required to produce liquid or molten cushioning material without being damaged or otherwise impaired. Preferably, the base member 210 is formed of a metal material.

Optionally, the cavity 230 is communicatively connected to a vacuum source (not shown) such that the cavity 230 can be placed under a vacuum at select times during the process for applying the cushioning material to the liner body 110. For example, a vacuum is preferably applied near or at the distal, closed end 233 of the cavity 230 to provide a force to assist the placement of the liner body 110 within the cavity 230. More specifically, one or more vacuum ports (not shown) are formed in the vertical housing 232 and in communication with the cavity 230 at or near the distal closed end 233 thereof. A conduit (e.g., tubing) can connect the vacuum ports to the vacuum source. When the vacuum source is actuated, air is withdrawn through the vacuum ports, thereby resulting in a pressure reduction in the distal end of the cavity 230. As will be described hereinafter, the liner body 110 is inserted into the cavity 230 such that the closed end 114 of the liner body 110 is inserted first and is positioned near or at the closed end 233 of the cavity 230. Because it is desirable to open up the liner body 110 so that it assumes a more tube-like shape, the actuation of the vacuum source causes the fabric material of the liner body 110 to be pulled toward the cavity wall of the cavity 230. The vacuum source thus serves to properly locate the liner body 110 within the cavity 230 (i.e., position and retain the closed distal end 114 of the liner body 110 at the closed end 233 of the cavity 230).

The mandrel 220 is an elongated structure having a first end 221 and a second end 222 that is received within the cavity 230 during the process of applying the cushion material to the interior 116 of the liner body 110. The exemplary mandrel 220 thus has a complementary shape relative to the cavity 230 so that at least a predetermined length of the mandrel 220 can be inserted into the cavity 230. In one exemplary embodiment, the mandrel 220 has a generally annular cross-sectional shape with the second end 222 terminating in a smooth rounded portion.

The cross-sectional dimensions of the mandrel 220 are preferably not uniform from the first end 221 to the second end 222. More specifically, the width of the mandrel 220 preferably varies along its length such that the mandrel 220 has a greater width near the first end 221 than at the second end 222. The mandrel 220 can thus be thought of as having an inward taper from the first end 221 towards the second end 222. While a number of different materials can be used to form the mandrel 220, the mandrel 220 must be formed of a material that can withstand the elevated temperatures of the liquid or molten cushioning material (e.g., 300°-400°). In one exemplary embodiment, the mandrel 220 is formed of a metal material.

The first end 221 of the mandrel 220 is connected to a base 223 that in turn can be connected to a larger sized support platform 225 that is preferably dimensioned so that it extends across the first plate 227.

The mandrel 220 is part of a system that includes a controller for adjusting the position of the mandrel 220 relative to the cavity 230. In one exemplary embodiment, the mandrel 220 is pneumatically operated by a programmable control unit that adjusts the position of the mandrel 220. In this embodiment, a pair of pneumatic cylinders 250 with drivable pistons 252 are disposed around the vertical housing 232 that includes the cavity 230. First ends of the pistons 252 are connected to the support platform 225 such that when the pistons 252 are pneumatically driven, the pistons are either extended or retracted within the cylinders 250, thereby causing relative movement of the support platform 225 and the mandrel 220 which is attached thereto. Because the mandrel 220 is axially aligned with the open end 231 of the cavity 230, the retraction of the pistons 252 causes the mandrel 220 to be drawn into the cavity 230.

Since the complete system is preferably a programmable computer controlled system, the precise coordinates of the mandrel 220 can easily be determined and monitored. Accordingly, computer controlled logic can be used to lower and retract the mandrel 220.

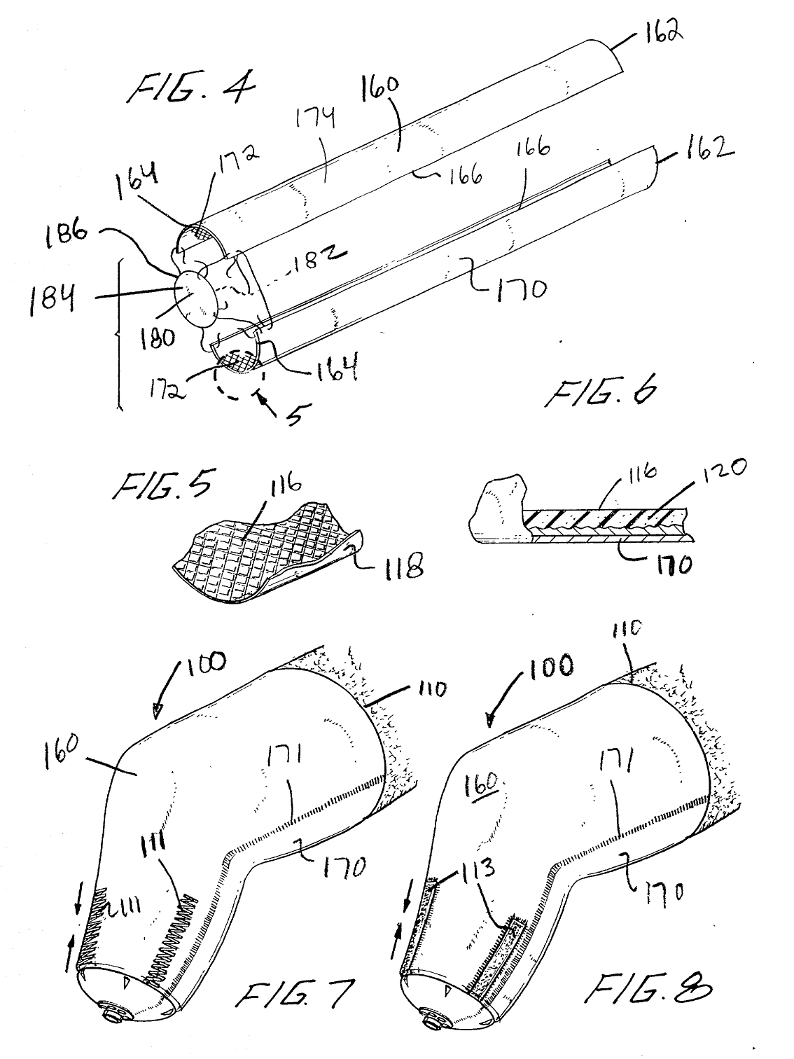

Referring to FIG. 3, the interior 116 of the liner body 110 is coated with the cushioning material in the following manner which ensures that the thickness of the cushioning material can be controlled with increase precision compared to conventional techniques. The liner body 110 is placed into the cavity 230 with the distal closed end 114 being inserted first into the cavity 230 and the liner body 110 is then properly located within the cavity, preferably by actuating the vacuum source to cause the liner body 110 to be drawn towards the cavity wall 236 of the cavity 230. In this manner, the liner body 110 assumes a more tube-like shape with the side panels 160, 170 be drawn apart from one another.

The cushioning material is heated to an elevated temperature that is above the point at which the cushioning material turns to a liquid or molten state and then the liquid or molten cushioning material is introduced into the inside of the liner body 110 by any number of conventional techniques, including injecting or otherwise feeding the cushioning material into the liner body 110. As previously-mentioned, the entire system is preferably a programmable, computer controlled system which is capable of determining the amount of cushioning material that needs to be introduced into the liner body 110 in order to provide a cushioning layer 120 having a predetermined thickness. The amount of cushioning material that is introduced is determined based upon a number of factors that are inputted into the system, including but not limited to, the dimensions of the liner body 110, the dimensions of the mandrel 220, the dimensions of the cavity 230, the desired thickness of the layer 120, etc.

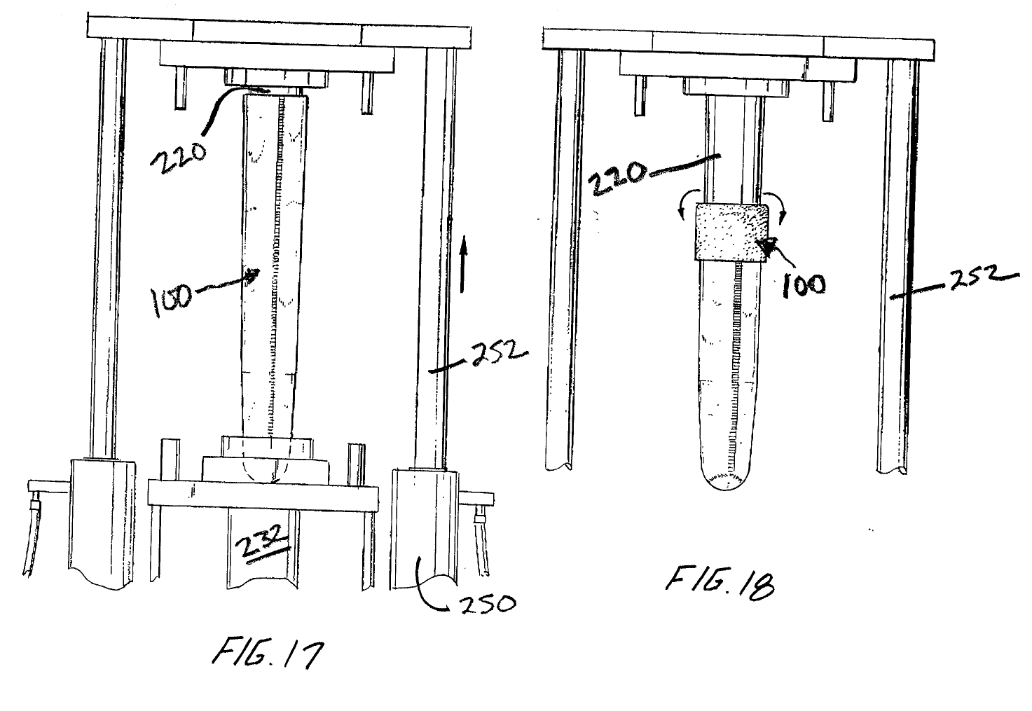

As the cushioning material is added, it will pool within the inside of the liner body 110 at the closed distal end 114 thereof as best shown in FIG. 13. The fabric material forming the liner body 110 is impermeable to the cushioning material and therefore, the cushioning material does not migrate from the interior 116 to the exterior 118. In order to effectively coat the inside of the liner body 110 to a predetermined thickness, the mandrel 220 is introduced inside of the liner body 110 (FIG. 14). The mandrel 220 is directed within the inside of the liner body 110 to a predetermined position where the distal end 222 of the mandrel 220 is disposed proximate to the closed distal end 114 of the liner body 110 (i.e., the distal end 222 is spaced a predetermined distance from the closed distal end 114). It will be appreciated that as the mandrel 220 is moved into this preselected position, the distal end 222 contacts the pool of cushioning material and as the mandrel 220 is further driven towards the closed distal end 114, the motion of the mandrel 220 causes the cushioning material to flow around the mandrel 220 as illustrated in FIGS. 15 and 16. In effect, the cushioning material is forced up the sides of the mandrel 220 between the mandrel 220 and the inside surface of the liner body 110. By carefully controlling the distance between the outer surface of the mandrel 220 and the interior surface of the liner body 110, the precise thickness of the cushioning layer 120 can be controlled. In other words, the thickness of the cushioning layer 120 will necessarily be the distance between the outer surface of the mandrel 220 and the interior surface of the liner body 110. However, the cushion material is slightly overpoured into the liner body 110 to ensure that a sufficient amount of cushion material is present to form the desired thickness of cushion layer 120.

The amount of cushioning material that is introduced into the inside of the liner body 110 is preferably determined beforehand so that cushioning material is not wasted when the mandrel 220 is introduced into the liner body 110 to cause the cushioning material to be dispersed around the mandrel 220. For example, if an excessive amount of cushioning material is disposed within the liner body 110, the action of the mandrel will cause the cushioning material to be discharged from the cavity 230 since there is an excessive amount of cushioning material to fit within the space between the outer surface of the mandrel 220 and the interior of the liner body 110.

The mandrel 220 can optionally include an extra safeguard to prevent the cushioning material from being discharged from the cavity 230. For example, the mandrel 220 can include a movable sleeve (not shown) at or near the end 221 of the mandrel 220 that is positionable between a retracted position and an extended position where the sleeve covers the gap between the mandrel and the interior surface of the liner body 110. In one exemplary embodiment, the movable sleeve is a spring-loaded ring that surrounds the mandrel 220 and is biased against the interior surface of the liner body 110. The spring-loaded ring serves to prevent the cushioning material from flowing out of the cavity 230 since it extends across and seals off the passage where the cushioning material flows. In another embodiment, a pneumatic controlled ring is used.

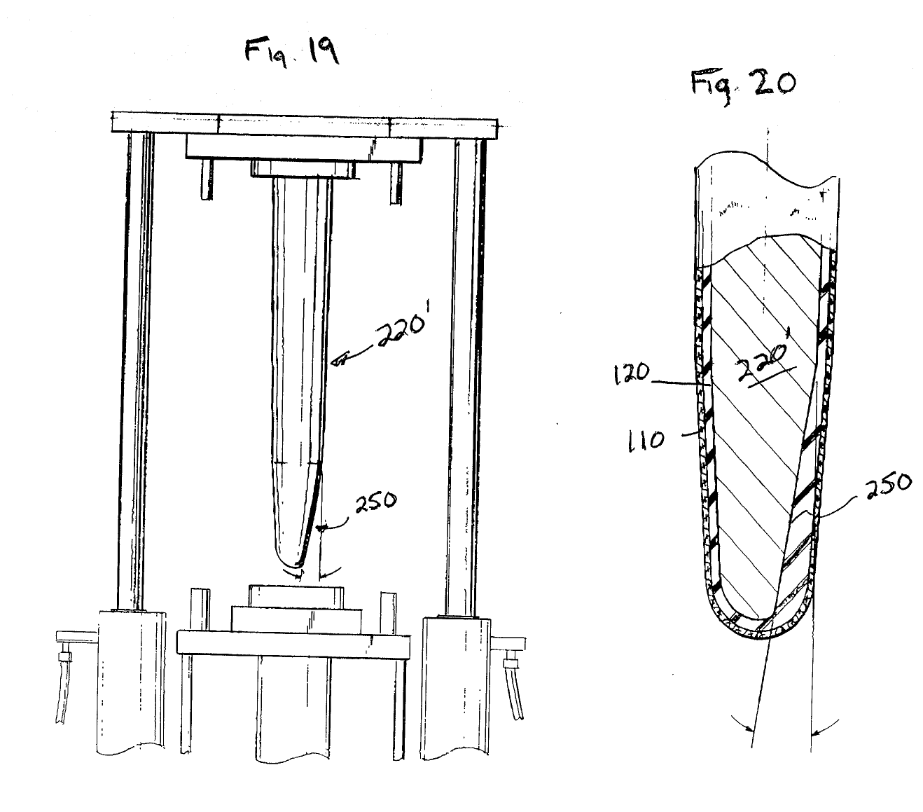

Once the mandrel 220 is driven to the preselected extended position to cause the cushioning material to flow around the mandrel 220, the cushioning material is permitted to cool for a predetermined amount of time. This results in the cushioning material solidifying into the gel-like cushion layer 120. After the cooling period has passed, the mandrel 220 is retracted from the cavity 230 with the cushioned liner 100 still being disposed over the mandrel 220 as illustrated in FIG. 17. The cushioned liner 100 is then removed from the mandrel 220 by rolling the cushioned liner 100 off of the mandrel 220 as illustrated in FIG. 18.

It will be appreciated that the application of a vacuum within the cavity 230 is not critical as it merely assists opening the liner body 110. This method of applying the cushioning material offers a number of advantages over conventional methods since it provides a simple yet effective method for controlling the thickness of the cushion layer 120 to a greater degree of precision compared to conventional techniques. Because the distance between the mandrel 220 and the interior of the liner body 110 can be determined for any point along the mandrel 220 and the liner body 110, the thickness of the cushion layer 120 is controlled since the thickness is defined by the distance between the mandrel 220 and the interior 116 of the liner body 110 when the exterior 118 of the liner body 110 is against the cavity wall 236 of the cavity 230.

It will also be appreciated that the inside contour of the cushion layer 120 (i.e., the interior 116 of the cushioned liner 100) is controlled by the contour of one or more of the mandrel 220 and the cavity 230. In one embodiment, the mandrel 220 is customized for a given application by altering the shape of the mandrel 220 along its longitudinal axis. For example, the mandrel 220 can include a recessed section along its length and when the mandrel 220 is introduced into the cavity 230 and the cushion material flows around the mandrel 220, the cushion material will settle within the recessed section of the mandrel 220. Because the distance between the recessed section and the liner body 110 is greater than the distance between surrounding portions of the mandrel 220 and the liner body 110, the thickness of the cushion layer 120 is greater in this section. Thus, a localized area of the interior 116 of the liner body 10 is formed to a greater thickness than surrounding sections. Preferably, this localized area of increased thickness is formed in an area where extra cushion is desired to provide additional comfort to the wearer as is the case in some of the sensitive interface locations between the residual limb 130 and the prosthetic device, etc. For example, the front portion of the cushioned liner 100 and distal end 114 are regions where additional comfort is desired since these regions (i.e., the shin area and distal stump area) correspond to sensitive areas of the residual limb 130. The amount of cushion material at the distal end 114 is controlled by the distal shape of the mandrel 220 and by the final position of the mandrel 220 relative to the distal end 114 of the liner body 110 (i.e., the greater the distance between the mandrel and the distal end 114, the greater the thickness of the cushion layer 120).

It will be appreciated that the mandrel 220 can be contoured in any number of different ways depending upon the desired contour of the cushion layer 120 of the cushioned liner 100. For example, the inward taper of the mandrel 220 can be non-uniform in manner to produce variations in thickness along the interior 116 of the cushioned liner 100. This embodiment is generally shown in FIGS. 19 and 20 in which a mandrel 220′ is illustrated. The mandrel 220′ is similar to the mandrel 220 with the exception that the distal end portion of the mandrel 220′ has a non-uniform cross-sectional shape. More specifically, one section of the distal end portion has a pronounced taper 250. In this section having a pronounced taper 250, the distance between the mandrel 220 and the liner body 110 is greater than in other surrounding sections and therefore, additional cushioning material is permitted to flow and ultimately cool and form the cushion layer 120 in this area. FIG. 20 clearly shows that the incorporation of the pronounced taper 250 into the mandrel 220 results in the cushioned liner 100 having one section (e.g., a front shin portion thereof) that has additional cushioning material provided thereat so as to provide the wearer with added comfort and protection in this sensitive area.

It will be appreciated that the mandrel 220 can be modified in any number of different ways to selectively control the thickness in localized areas of the cushioned liner 100. For example, the distal end 222 of the mandrel 220 can have an annular recessed section that receives additional cushioning material, thereby altering the thickness profile at the distal end 114 of the cushioned liner 100.

If an area of reduced thickness is desired, the mandrel 220 can be modified to include one or more protruding features (i.e., a section of the outer surface of the mandrel 220 that protrudes outwardly relative to the surrounding sections of the mandrel 220). Each protruding feature reduces the distance between the mandrel 220 and the interior of the liner body 110 and thus causes the cushion layer 120 to have a reduced thickness in this region.

In another aspect, the cavity 230 does not necessarily have to have a uniform shape; but rather, the cavity 230 can be formed to have any number of shapes. By altering the contour of the cavity 230, the thickness of the cushion layer 120 of the cushioned liner 100 can be varied in select locations. For example, the cavity wall of the cavity 230 can include a recessed section or a protruding section to cause the liner body 110 assume a different shape in this section.

Thus, it will be understood that the present method permits a number of different cushioned liners 100 to be produced using a limited amount of equipment since the mandrel 220 is interchangeable and one mandrel 220 having one profile can be interchanged for another mandrel 220 having another profile. By interchanging the mandrel 220 and maintaining the same cavity 230, the thickness and contour of the cushion layer 120 can be controlled to a high degree of precision. Further, the present method is less complicated than the conventional methods, including the dipping method, as it does not require a series of steps to build-up the cushion layer 120 to the desired thickness nor does it require that the cushioned liner 100 be inverted after the cushion layer 120 is formed on the interior of the liner body 110.

The present application thus provides cushioned liners that advantageously are constructed so that the interface between the residual limb and the prosthetic device is improved by eliminating the distal seam that extends across the sensitive distal end of the residual limb. Further, the present method permits the thickness of the cushion layer to be controlled with enhanced precision and also permits the profile (contour) of the cushion layer to be readily changed.

While the invention has been particularly shown and described with reference to preferred embodiments thereof, it will be understood by those skilled in the art that various changes in form and details may be made therein without departing from the spirit and scope of the invention.

What is claimed is:

1. A cushioned liner for covering a residual limb of an amputee, the cushioned liner comprising:
a sock-shaped fabric member formed of at least two fabric pieces, one of the fabric pieces being a distal end piece that is attached to at least one other fabric piece along a circumferential edge of the distal end piece, the distal end piece being free of a transverse seam extending across the distal end piece; and
a cushion layer disposed on at least one side of the sock-shaped fabric member.

2. The cushioned liner of claim 1, wherein the sock-shaped fabric member is formed of first and second side pieces that are attached to one another along vertical edges thereof, the distal end piece having an annular shape such that the distal end piece is attached to one end of each of the first and second side pieces to produce a circumferential seam.

3. The cushioned liner of claim 2, wherein each of the first and second side pieces has an elongated generally rectangular shape.

4. The cushioned liner of claim 2, wherein the first and second side pieces are stitched to one another along the vertical edges thereof.

5. The cushioned liner of claim 4, wherein the stitch is-of a flat-locked stitch type.

6. The cushioned liner of claim 2, wherein the distal end piece is attached to the first and second side pieces by a circumferential stitched seam.

7. The cushioned liner of claim 6, wherein the circumferential stitched seam is of a flat-locked stitch type.

8. The cushioned liner of claim 1, wherein the cushion layer has a uniform thickness profile.

9. The cushioned liner of claim 1, wherein the cushion layer has an uneven thickness profile.

10. The cushioned liner of claim 1, wherein the sock-shaped fabric member is formed of a fabric material that is impermeable to the cushion layer so that the material forming the cushion layer is prevented from migrating from one side of the fabric material to an opposite side of the fabric material.

11. The cushioned liner of claim 1, wherein the sock-shaped fabric member is formed of at least two polymeric materials.

12. The cushioned liner of claim 11, wherein the sock-shaped fabric member is formed of a knit of polyester fibers and polypropylene fibers, the polyester fibers formed a first side of the sock-shaped fabric member and the polypropylene fibers forming a second side of the sock-shaped fabric member.

13. The cushioned liner of claim 12, wherein the cushion layer is formed on the second side of the sock-shaped fabric member.

14. The cushioned liner of claim 1, wherein the cushion layer is formed on interior surfaces of the sock-shaped fabric member.

15. The cushioned liner of claim 1, wherein the cushion layer is formed of a gel composition.

16. The cushioned liner of claim 15, wherein the gel composition comprises a block copolymer and mineral oil.

17. The cushioned liner of claim 16, wherein the block copolymer is a polystyrene-poly(ethylene-ethylene/propylene)-polystyrene (SEEPS) block copolymer.

18. The cushioned liner of claim 1, further including:
a stretch limiting feature incorporated into the sock-shaped fabric member at a distal end portion thereof.

19. The cushioned liner of claim 18, wherein the stretch limiting feature comprises a member that is formed of a material that has limited stretch characteristics such that when the stretch limiting feature is incorporated into the sock-shaped fabric member, the elasticity of the distal end portion is reduced.

20. The cushioned liner of claim 18, wherein the stretch limiting feature comprises one of embroidery integrated into the distal end portion and a strap formed of a material having low elasticity.

21. The cushioned liner of claim 20, wherein the stretch limiting feature is confined to an area occupying a distalmost 3 to 7 inches of the fabric member.

22. The cushioned liner of claim 20, wherein the sock-shaped fabric member is formed of first and second side pieces that are attached to one other along vertical edges thereof, the distal end piece having an annular shape such that the distal end piece is attached to one end of each of the first and second side pieces to produce a circumferential seam, the strap being attached to the first and second side pieces along one vertical seam that joins adjacent vertical sides to one another.

23. A cushioned liner comprising:
a sock-shaped fabric member formed of at least two fabric pieces, one of the fabric pieces being a distal end piece that is stitched to at least one other fabric piece only along a circumferential seam formed at a peripheral edge of the distal end piece; and
a cushion layer disposed on at least one side of the sock-shaped fabric member.

24. The cushioned liner of claim 23, wherein the cushion layer is formed of a gel composition.

25. The cushioned liner of claim 24, wherein the gel composition comprises a block copolymer and mineral oil.

26. The cushioned liner of claim 25, wherein the block copolymer is a polystyrene-poly(ethylene-ethylene/propylene)-polystyrene (SEEPS) block copolymer.

27. The cushioned liner of claim 23, wherein the sock-shaped fabric member is formed of first and second side pieces that are attached to one another along vertical edges thereof, the distal end piece having an annular shape such that the distal end piece is attached to one end of each of the first and second side pieces to produce a circumferential seam.

28. The cushioned liner of claim 27, wherein the first and second side pieces are stitched to one another along the vertical edges thereof, thereby forming vertical seams.

29. The cushioned liner of claim 23, wherein the sock-shaped fabric member is formed of a fabric material that is impermeable to the cushion layer so that the material forming the cushion layer is prevented from migrating from one side of the fabric material to an opposite side of the fabric material.

30. The cushioned liner of claim 23, wherein the sock-shaped fabric member is formed of at least two polymeric materials.

31. The cushioned liner of claim 30, wherein the sock-shaped fabric member is formed of a knit of polyester fibers and polypropylene fibers, the polyester fibers formed a first side of the sock-shaped fabric member and the polypropylene fibers forming a second side of the sock-shaped fabric member.

32. A fabric liner adapted to receive a cushioning material to form a cushioned liner for covering a residual limb of an amputee, the fabric liner comprising:
a sock-shaped fabric body formed of at least two fabric pieces, one of the fabric pieces being a distal end piece that is attached to at least one other fabric piece along a circumferential edge of the distal end piece, the distal end piece being free of a transverse seam extending across the distal end piece.

33. The fabric liner of claim 32, wherein the sock-shaped fabric body is formed of first and second side pieces that are attached to one another along vertical edges thereof, the distal end piece having an annular shape such that the distal end piece is attached to one end of each of the first and second side pieces to produce a circumferential seam.

34. The fabric liner of claim 33, wherein the first and second side pieces are stitched to one another along the vertical edges thereof.

35. The fabric liner of claim 34, wherein the stitch is of a flat-locked stitch type.

36. The fabric liner of claim 33, wherein the distal end piece is attached to the first and second side pieces by a circumferential stitched seam.

37. The fabric liner of claim 36, wherein the circumferential stitched seam is of a flat-locked stitch type.

38. The fabric liner of claim 33, wherein the sock-shaped fabric body is formed of at least two polymeric materials.

39. The fabric liner of claim 32, wherein the sock-shaped fabric body is formed of a knit of polyester fibers and polypropylene fibers, the polyester fibers formed a first side of the sock-shaped fabric body and the polypropylene fibers forming a second side of the sock-shaped fabric body.

40. The fabric liner of claim 32, further including:
a stretch limiting feature incorporated into the sock-shaped fabric body at a distal end portion thereof.

41. The fabric liner of claim 40, wherein the stretch limiting feature comprises a member that is formed of a material that has limited stretch characteristics such that when the stretch limiting feature is incorporated into the sock-shaped fabric body, the elasticity of the distal end portion is reduced.

42. The fabric liner of claim 40, wherein the stretch limiting feature comprises one of embroidery integrated into the distal end portion and a strap formed of a material having low elasticity.

43. The fabric liner of claim 40, wherein the stretch limiting feature is confined to an area occupying a distalmost 3 to 7 inches of the fabric sleeve body.

44. The fabric liner of claim 40, wherein the sock-shaped fabric body is formed of first and second side pieces that are attached to one other along vertical edges thereof, the distal end piece having an annular shape such that the distal end piece is attached to one end of each of the first and second side pieces to produce a circumferential seam, the strap being attached to the first and second side pieces along one vertical seam that joins adjacent vertical sides to one another.

45. A fabric liner used in constructing a cushioned prosthetic liner, the fabric liner comprising:
a fabric body formed of at least two fabric pieces, one of the fabric pieces being a distal end piece that is attached to at least one other fabric piece by only a circumferential stitched seam formed at a peripheral edge of the distal end piece.
